(19) United States
(12) Patent Application Publication
(10) Pub. No.: US 2026/0048279 A1
(43) Pub. Date: Feb. 19, 2026

(54) VEHICLE OCCUPANT CARE DEVICE AND METHOD

(71) Applicants: Hyundai Motor Company, Seoul (KR); Kia Corporation, Seoul (KR)

(72) Inventors: Ki Chang KIM, Hwaseong-Si (KR); Dong Chul PARK, Hwaseong-Si (KR); Eun Soo JO, Hwaseong-Si (KR); Jeong Goo KANG, Hwaseong-Si (KR)

(73) Assignees: Hyundai Motor Company, Seoul (KR); Kia Corporation, Seoul (KR)

(21) Appl. No.: 19/048,241

(22) Filed: Feb. 7, 2025

(30) Foreign Application Priority Data
Aug. 16, 2024 (KR) 10-2024-0109638

(51) Int. Cl. A61N 7/00; B60W 50/00; G06V 20/59; G06V 40/16

(52) U.S. Cl. CPC A61N 7/00; B60W 50/0098; G06V 20/59; G06V 40/161; A61N 2007/0034; B60W 2420/403; B60W 2420/54; B60W 2520/10; B60W 2540/049

(57) ABSTRACT

A vehicle occupant care device includes a communication unit configured to collect traveling information of a vehicle, a camera configured to photograph a vehicle occupant and generate image data, a first processing unit configured to determine a face position of the occupant using the image data, a second processing unit configured to output a first ultrasonic radiation signal when the vehicle stops and output a second ultrasonic radiation signal when the vehicle travels according to the traveling information, and an ultrasonic radiation unit fixed to the interior of the vehicle and configured to output an ultrasonic wave to the face position of the occupant according to the first ultrasonic radiation signal or the second ultrasonic radiation signal.

### CROSS-REFERENCE TO RELATED APPLICATION

The present application claims priority to Korean Patent Application No. 10-2024-0109638, filed on Aug. 16, 2024, the entire contents of which is incorporated herein for all purposes by this reference.

### BACKGROUND OF THE PRESENT DISCLOSURE

### Field of the Present Disclosure

The present disclosure relates to a vehicle occupant care device and method.

### Description of Related Art

Skin care solutions are generally implemented in cosmetics stores, care centers, or at home. General skin care solution methods are implemented with mechanical methods in parallel with the help of experts in stores or rooms.

However, it is difficult for conventional techniques implemented by experts in cosmetics stores or rooms to be applied to occupants in vehicles. When a vehicle occupant is in a driving state, it is difficult for the occupant to manipulate a skin care device, and when the occupant manipulates the skin care device while driving, it may be a threat to safe driving.

The information included in this Background of the present disclosure is only for enhancement of understanding of the general background of the present disclosure and may not be taken as an acknowledgement or any form of suggestion that this information forms the prior art already known to a person skilled in the art.

### BRIEF SUMMARY

Various aspects of the present disclosure are directed to providing a vehicle occupant care device and method, which may provide a skin care solution for a vehicle occupant.

According to an exemplary embodiment of the present disclosure, there is provided a vehicle occupant care device including a communication unit configured to collect traveling information of a vehicle, a camera configured to photograph a vehicle occupant and generate image data, a first processing unit configured to determine a face position of the occupant using the image data, a second processing unit configured to output a first ultrasonic radiation signal when the vehicle stops and output a second ultrasonic radiation signal when the vehicle travels according to the traveling information, and an ultrasonic radiation unit fixed to the interior of the vehicle and configured to output an ultrasonic wave to the face position of the occupant according to the first ultrasonic radiation signal or the second ultrasonic radiation signal.

The first output may have a lower ultrasonic signal intensity than the second output.

The second processing unit may differently set an ultrasonic output position of the first ultrasonic radiation signal and an ultrasonic output position of the second ultrasonic radiation signal.

The first ultrasonic radiation signal may have a fixed frequency value.

The second ultrasonic radiation signal may have a variable frequency value.

The first ultrasonic signal may have a composite modulation waveform (multi sweep control).

The second processing unit may output a 2-1 ultrasonic radiation signal when the vehicle travels at a constant speed within a preset speed range.

The second processing unit may output a 2-2 ultrasonic radiation signal when the vehicle performs gear shifting and travels at a speed out of the speed range.

The 2-2 ultrasonic radiation signal may have a lower ultrasonic signal intensity than the 2-1 ultrasonic radiation signal.

The 2-1 ultrasonic radiation signal may have a double side band (DSB) waveform (sweep control).

The 2-2 ultrasonic radiation signal may have a single side band (SSB) waveform (sweep).

The first processing unit may be configured to determine the number of vehicle occupants using the image data, and the second processing unit may adjust at least one of an intensity, a frequency, and an ultrasonic output position of the ultrasonic radiation signal according to the number of vehicle occupants.

The communication unit may collect tilt information and pressure information of a seat on which the occupant sits.

The second processing unit may be configured to determine the face position of the occupant using the image data, and the tilt information and the pressure information of the seat.

According to another exemplary embodiment of the present disclosure, there is provided a vehicle occupant care method including collecting, by a communication unit, traveling information of a vehicle, photographing, by a camera, a vehicle occupant and generating image data, determining, by a first processing unit, a face position of the occupant using the image data, determining, by a second processing unit, whether the vehicle stops based on the traveling information, outputting, by the second processing unit, a first ultrasonic radiation signal when the vehicle stops or outputting a second ultrasonic radiation signal when the vehicle travels, and outputting, by an ultrasonic radiation unit fixed to the interior of the vehicle, an ultrasonic wave to the face position of the occupant according to the first ultrasonic radiation signal or the second ultrasonic radiation signal.

The first output may have a lower ultrasonic signal intensity than the second output.

The outputting of the second ultrasonic radiation signal may include determining, by the second processing unit, whether the vehicle travels at a constant speed, and outputting, by the second processing unit, a 2-1 ultrasonic radiation signal when the vehicle travels at a constant speed.

The vehicle occupant care method may further include outputting, by the second processing unit, a 2-2 ultrasonic radiation signal when the vehicle performs gear shifting and travels.

The 2-2 ultrasonic radiation signal may have a lower ultrasonic signal intensity than the 2-1 ultrasonic radiation signal.

The vehicle occupant care method may further include determining, by the first processing unit, the number of vehicle occupants using the image data, and adjusting, by the second processing unit, at least one of an intensity, a frequency, and an ultrasonic output position of the ultrasonic radiation signal according to the number of vehicle occupants.

The methods and apparatuses of the present disclosure have other features and advantages which will be apparent from or are set forth in more detail in the accompanying drawings, which are incorporated herein, and the following Detailed Description, which together serve to explain certain principles of the present disclosure.

### BRIEF DESCRIPTION OF THE DRAWINGS

FIG. 1 is a conceptual diagram of a vehicle occupant care device according to an exemplary embodiment of the present disclosure;

FIG. 2 is a block diagram of the vehicle occupant care device according to the embodiment;

FIG. 3 is a view for describing an ultrasonic radiation unit according to an exemplary embodiment of the present disclosure;

FIG. 4, FIG. 5, FIG. 6, FIG. 7, FIG. 8, FIG. 9, FIG. 10 and FIG. 11 are views for describing an operation of a second processing unit according to an exemplary embodiment of the present disclosure; and

FIG. 12, FIG. 13 and FIG. 14 are flowcharts of a vehicle occupant care method according to an exemplary embodiment of the present disclosure.

It may be understood that the appended drawings are not necessarily to scale, presenting a somewhat simplified representation of various features illustrative of the basic principles of the present disclosure. The specific design features of the present disclosure as included herein, including, for example, specific dimensions, orientations, locations, and shapes will be determined in part by the particularly intended application and use environment.

In the figures, reference numbers refer to the same or equivalent portions of the present disclosure throughout the several figures of the drawing.

### DETAILED DESCRIPTION

Reference will now be made in detail to various embodiments of the present disclosure(s), examples of which are illustrated in the accompanying drawings and described below. While the present disclosure(s) will be described in conjunction with exemplary embodiments of the present disclosure, it will be understood that the present description is not intended to limit the present disclosure(s) to those exemplary embodiments of the present disclosure. On the other hand, the present disclosure(s) is/are intended to cover not only the exemplary embodiments of the present disclosure, but also various alternatives, modifications, equivalents and other embodiments, which may be included within the spirit and scope of the present disclosure as defined by the appended claims.

Hereinafter, various exemplary embodiments of the present disclosure will be described in detail with reference to the accompanying drawings.

However, the technical spirit of the present disclosure is not limited to various exemplary embodiments which will be described and may be implemented in a variety of different forms, and one or more components of the exemplary embodiments of the present disclosure may be selectively combined, substituted, and used within the range of the technical spirit of the present disclosure.

Furthermore, unless clearly and specifically defined otherwise by the context, all terms (including technical and scientific terms) used herein can be interpreted as having meanings customarily understood by those skilled in the art, and the meanings of generally used terms, such as those defined in commonly used dictionaries, will be interpreted based on contextual meanings of the related art.

Furthermore, the terms used in the exemplary embodiments of the present disclosure are considered in a descriptive sense only and not to limit the present disclosure.

In the present specification, unless specifically indicated otherwise by the context, singular forms include plural forms, and in a case in which “at least one (or one or more) among A, B, and C” is described, this may include at least one combination among all possible combinations of A, B, and C.

Furthermore, in descriptions of components of the present disclosure, terms such as “first,” “second,” “A,” “B,” “(a),” and “(b)” may be used.

The terms are only to distinguish one component from another component, and the essence, order, and the like of the components are not limited by the terms.

Furthermore, it should be understood that, when a first component is referred to as being “connected” or “coupled” to a second component, such a description may include both a case in which the first component is directly connected or coupled to the second component, and a case in which the first component is connected or coupled to the second component with a third component disposed therebetween.

Furthermore, when a first component is described as being formed or disposed “on” or “under” a second component, such a description includes both a case in which the two components are formed or disposed in direct contact with each other and a case in which one or more other components are interposed between the two components. In addition, when the first component is described as being formed “on or under” the second component, such a description may include a case in which the first component is formed at an upper side or a lower side with respect to the second component.

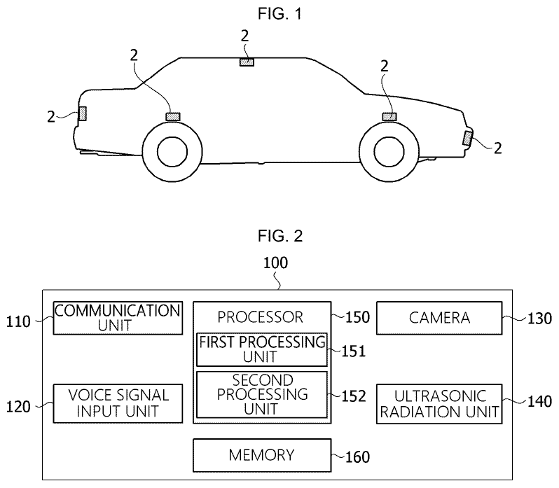

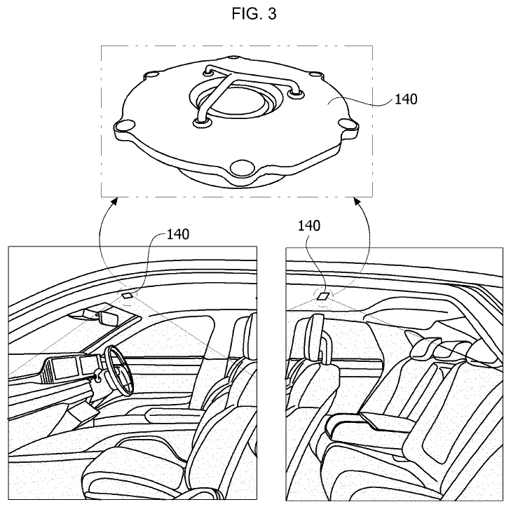

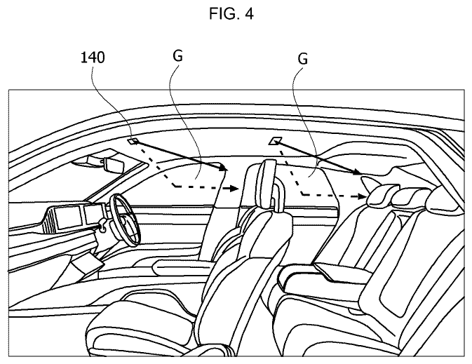

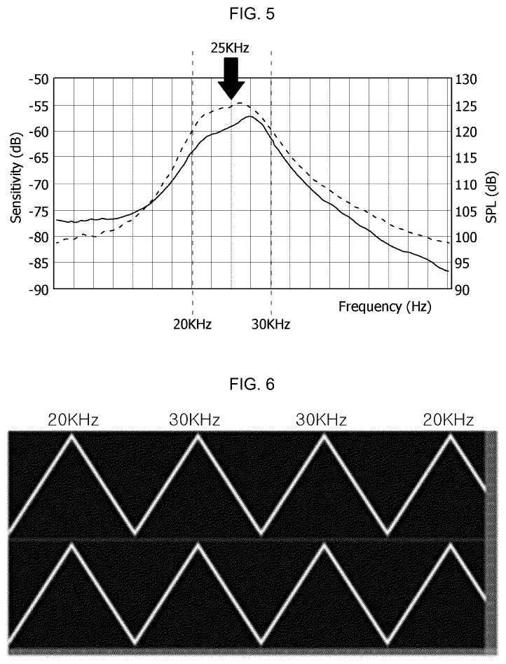

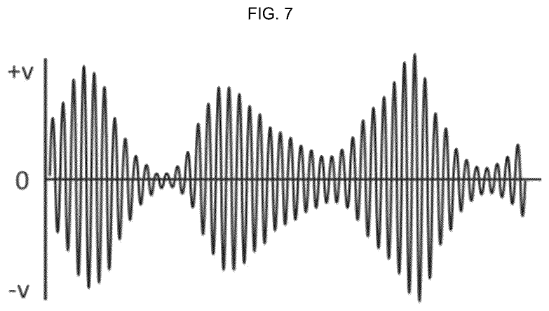

FIG. 1 is a conceptual diagram of a vehicle occupant care device according to an exemplary embodiment of the present disclosure, and FIG. 2 is a block diagram of the vehicle occupant care device according to the exemplary embodiment of the present disclosure. Referring to FIG. 1 and FIG. 2, a vehicle occupant care device 100 according to the exemplary embodiment of the present disclosure may include a communication unit 110, a voice signal input unit 120, a camera 130, an ultrasonic radiation unit 140, a processor 150, and a memory 160.

A vehicle 1 according to the exemplary embodiment of the present disclosure may further include other components in addition to components described herein or may not include some of the described components. Various detection elements mounted on the vehicle 1 may detect a state of the vehicle. Detection elements 2 may include an attitude sensor (e.g., a yaw sensor, a roll sensor, or a pitch sensor), a collision sensor, a wheel sensor, a speed sensor, an inclination sensor, a weight detection sensor, a heading sensor, a gyro sensor, a position module, a vehicle forward/backward movement sensor, a battery sensor, a fuel sensor, a tire sensor, a steering sensor by steering wheel rotation, a vehicle interior temperature sensor, a vehicle interior humidity sensor, an ultrasonic sensor, an illuminance sensor, an accelerator pedal position sensor, a brake pedal position sensor, a seat tilt sensor, seat pressure information, etc.

The detection element 2 may obtain detection signals for vehicle attitude information, vehicle collision information, vehicle direction information, vehicle position information (global positioning system (GPS) information), vehicle angle information, vehicle speed information, vehicle acceleration information, vehicle angle information, vehicle forward/backward movement information, battery information, fuel information, tire information, vehicle lamp information, vehicle interior temperature information, vehicle interior humidity information, steering wheel rotation angle, vehicle external illuminance, a pressure applied to an accelerator pedal, a pressure applied to a brake pedal, etc.

The detection element 2 may further include an accelerator pedal sensor, a pressure sensor, an engine rotation speed sensor, an air flow sensor (AFS), an intake air temperature sensor (ATS), a water temperature sensor (WTS), a throttle position sensor (TPS), a top dead center portion (TDC) sensor, a crank angle sensor (CAS), etc.

The detection element 2 may be configured to generate vehicle state information based on sensing data. The vehicle state information may be information generated based on data detected by various detection elements provided inside the vehicle.

For example, the vehicle state information may include vehicle attitude information, vehicle speed information, vehicle angle information, vehicle weight information, vehicle direction information, vehicle battery information, vehicle fuel information, vehicle tire pressure information, vehicle steering information, vehicle interior temperature information, vehicle interior humidity information, pedal position information, vehicle engine temperature information, seat tilt information, seat pressure information, etc.

In an exemplary embodiment of the present disclosure, each component may have different functions and capabilities in addition to the above and include additional components in addition to those to be described below. Furthermore, in an exemplary embodiment of the present disclosure, each component may be implemented using one or more physically separate devices, or implemented by one or more processors 150 or a combination of the one or more processors 150 and software, and may not be clearly distinguished in specific operations unlike the shown example.

The vehicle occupant care device 100 according to the exemplary embodiment of the present disclosure may be implemented in a logic circuit by hardware, firmware, software, or a combination thereof and implemented using a general-purpose or special-purpose computer. The device may be implemented using a hardwired device, a field programmable gate array (FPGA), an application specific integrated circuit (ASIC), etc. Furthermore, the device may be implemented as a system on chip (SoC) including the one or more processors 150 and a controller.

Furthermore, the vehicle occupant care device may be mounted on a computing device or server provided with hardware elements in a form of software, hardware, or a combination thereof. The computing device or the server may be various devices including all or some of a communication device such as a communication modem for performing communication with various devices or a wired/wireless communication network, the memory 160 for storing data for executing a program, a microprocessor for executing a program to perform determinations and instructions, etc.

The communication unit 110 may collect vehicle traveling information and collect tilt information and pressure information of a seat on which an occupant sits.

In an exemplary embodiment of the present disclosure, the vehicle traveling information may include vehicle position information (GPS information), vehicle angle information, vehicle speed information, and vehicle acceleration information.

The communication unit 110 may support the vehicle occupant care device to communicate with electronic control units (ECUs) mounted on the vehicle. The communication unit 110 may include a transceiver for transmitting and receiving Controller Area Network (CAN) messages using a CAN protocol. The communication unit 110 may also support the vehicle occupant care device to communicate with external electronic devices (e.g., a terminal and a server). The communication unit 110 may include a wireless communication circuit and/or a wired communication circuit.

The memory 160 may include a database (DB). The memory 160 may be a non-transitory storage medium for storing instructions executed by a first processing unit 151 and a second processing unit 152. The memory 160 may include at least one of a random access memory (RAM), a static random access memory (SRAM), a read only memory (ROM), a programmable read only memory (PROM), an electrically erasable PROM (EEPROM), an erasable PROM (EPROM), a Hard Disk Drive (HDD), a solid state disk (SSD), an embedded multimedia card (eMMC), a universal flash storage (UFS), and/or a web storage.

In an exemplary embodiment of the present disclosure, the first processing unit 151 and the second processing unit 152 may be implemented through the same process, and for the convenience of description, an operation of each component will be described separately below.

The processor 150 may include at least one of processing devices such as an ASIC, a digital signal processor (DSP), a programmable logic device (PLD), an FPGA, a central processing unit (CPU), a microcontroller, and/or a microprocessor.

The voice signal input unit 120 may be mounted in the vehicle interior and may detect an occupant's voice to generate a voice signal. The voice signal input unit 120 may be configured as a microphone and may receive the vehicle occupant's voice. When receiving the occupant's voice, the voice signal input unit may convert the voice in a form of a sound wave into an audio signal, which is an electrical signal. Therefore, the occupant's voice signal generated by the voice signal input unit 120 may be an electrical audio signal.

The voice signal input unit 120 may detect a mode selection instruction of the occupant and transmit the same to the processor 150. The mode selection instruction of the occupant may be a voice for selecting a rest mode, a sleep mode, or a skin care mode, and the voice signal input unit 120 may convert the mode selection instruction of the occupant into a voice signal and transmit the voice signal to the processor 150.

In an exemplary embodiment of the present disclosure, when receiving a voice signal of the rest mode or the sleep mode through the voice signal input unit 120, the processor 150 may be configured for controlling the ultrasonic radiation unit 140 to output an ultrasonic signal to the vehicle occupant.

The camera 130 may photograph the vehicle occupant and generate image data related to the vehicle occupant. The camera 130 may include an image sensor disposed to face an occupant in the vehicle interior. The camera 130 may capture a front image of the vehicle occupant according to a preset cycle and transmit the captured front image to the processor 150. In an exemplary embodiment of the present disclosure, the front image of the occupant may be captured to include the entire upper body of the vehicle occupant. A direction in which a lens of the camera 130 faces may be adjusted under the control of the processor 150.

The camera 130 may be an image sensor for photographing a subject using a complementary metal-oxide semiconductor (CMOS) module, a charge coupled device (CCD) module, etc. In the instant case, the input image frame is provided to the CMOS module or the CCD module in the camera 130 through the lens, and the CMOS module or the CCD module converts an optical signal of the subject passing through the lens into an electrical signal (image data) and outputs the electrical signal (image data).

The camera 130 may include a fish-eye lens or a wide-angle lens with a wide field of view. Therefore, the camera 130 may photograph the entire upper body of the occupant.

In an exemplary embodiment of the present disclosure, the image data may be consecutive image frames composed of N×M pixels (N and M are natural numbers).

The ultrasonic radiation unit 140 may be fixed to a vehicle interior roof trim to output ultrasonic waves to the occupant's face position according to a first ultrasonic radiation signal or a second ultrasonic radiation signal under the control of the processor 150.

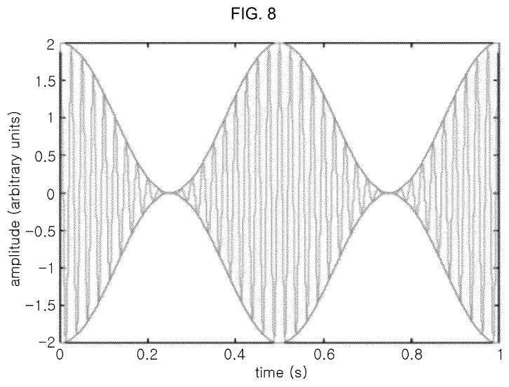

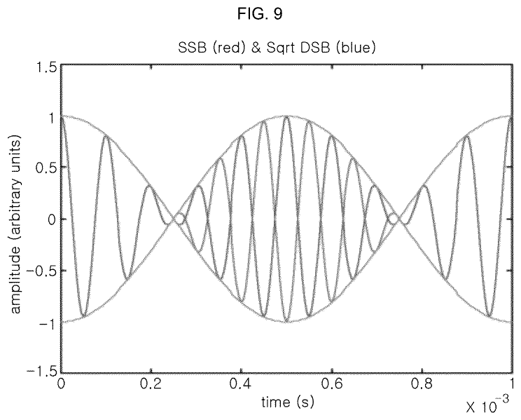

FIG. 3 is a view for describing an ultrasonic radiation unit according to an exemplary embodiment of the present disclosure. Referring to FIG. 3 together, the ultrasonic radiation unit 140 may be fixed to a front portion of the vehicle roof trim and a center portion of the roof trim to output ultrasonic waves toward the vehicle interior. The ultrasonic radiation unit 140 may be fixed to an indoor ceiling of the vehicle in a form of a circular tweeter and provided with a mechanical mechanism configured for 360 degrees rotation to change an orientation under the control of the processor.

The ultrasonic radiation unit 140 may be configured to generate ultrasonic waves using the reverse piezoelectric effect, which generates ultrasonic waves by applying an AC current to a crystal of a material and then causing the crystal to repeatedly compress and expand, generating mechanical vibrations. The ultrasonic radiation unit 140 generates ultrasonic waves by converting a high-frequency AC current into mechanical vibrations by the reverse piezoelectric effect.

The ultrasonic radiation unit 140 according to the exemplary embodiment of the present disclosure may be fixed to the vehicle roof trim to output ultrasonic waves toward the vehicle interior. The ultrasonic radiation unit 140 may be fixed to the indoor ceiling of the vehicle in a form of the circular tweeter, and a mechanical mechanism configured for 360 degrees rotation may be provided to change the orientation under the control of the processor 150.

The first processing unit 151 may be configured to determine a face position of the occupant using the image data.

The first processing unit 151 may extract feature points in a facial portion of the occupant from the image data to determine a facial region and analyze the yaw, pitch, and roll of the face based on the extracted feature points to determine the occupant's face position and orientation.

Alternatively, the first processing unit 151 may be configured to determine the occupant's face position using a human pose estimation method. For example, the first processing unit 151 may detect the occupant's facial region by cropping an image connecting a head and a neck from the image data using the human pose estimation method.

In an exemplary embodiment of the present disclosure, the human pose estimation method is an algorithm which is configured to detect a position and orientation of an object in computer vision and may localize and estimate positions at which key points, which are human body joints, are configured. The main key points of the body may include a head, a neck, a shoulder, an elbow, a wrist, a hip, a pelvis, a knee, an ankle, etc. The first processing unit may detect a person from the image data, estimate a pose inside a bounding box, and then crop the occupant's facial region as a key point portion.

Alternatively, the first processing unit 151 may detect an outline of an object from the image data, compare the detected outline with a person's facial portion that has been previously stored in a database, and detect a region with an outline that matches the pre-stored person's facial portion as the occupant's facial region.

Furthermore, for example, the first processing unit 151 may extract a feature point of an object in the image data and when the extracted feature point matches a feature point of the person's facial portion that has been previously stored in the database with a proximity greater than or equal to a threshold value, detect the corresponding region as the occupant's facial region. In the instant case, the first processing unit may extract feature points from the pieces of image data of two objects to be compared and use a scale invariant feature transform (SIFT) or speeded up robust features (SURF) algorithm that matches feature point descriptors of the two extracted objects.

Furthermore, for example, the first processing unit 151 may detect the occupant's facial region based on outlines of the objects from the image data. The first processing unit 151 may detect the outlines of the objects from the image data to generate an edge image, detect the outlines from foreground image data, which is an image of the vehicle interior stored in advance in the database, to generate a background edge image, and detect the occupant's facial region from a different image obtained by subtracting the background edge image from the edge image. In the instant case, the first processing unit 151 detects the outline of the object appearing in the frame as an edge using gradient information of an image data frame to generate the edge image. Here, the gradient information is a value generated from a difference value between adjacent pixels among predetermined pixels in the frame and means the sum of absolute values of the differences, and the edge is a boundary line between objects using the gradient information.

Furthermore, the first processing unit 151 may detect the occupant's facial region from the image using an object detection classifier. In the instant case, the object detection classifier is trained using a training DB which is built using images previously captured by changing different postures of the occupant or external environments, and the object detection classifier generates a DB for the occupant's facial portion through various learning algorithms including a support vector machine (SVM), a neural network, and an AdaBoost algorithm. The first processing unit 151 may detect an edge of an object corresponding to the foreground in the previously captured background image of the vehicle, apply the edge of the foreground object detected from the image data, and apply the object detection classifier to the region of the image data to which the edge of the foreground object is applied to detect the occupant's facial region.

Furthermore, the first processing unit 151 may reduce noise in the image data captured by the camera 130 and perform image signal processing for improving image quality, such as gamma correction, color filter array interpolation, color matrix, color correction, and color enhancement. Furthermore, the first processing unit 151 may also perform color processing, blur processing, edge emphasis processing, image interpretation processing, image recognition processing, image effect processing, etc.

Furthermore, the first processing unit 151 may be configured to determine the number of vehicle occupants using the image data. The first processing unit 151 may detect the facial region from the image data according to the above-described method and determine the number of vehicle occupants according to numbers of detected facial regions.

The second processing unit 152 may output the first ultrasonic radiation signal when the vehicle stops and output the second ultrasonic radiation when the vehicle travels according to traveling information. The second processing unit 152 may transmit an ultrasonic radiation signal to the ultrasonic radiation unit 140, and the ultrasonic radiation unit 140 may output ultrasonic waves having a specific intensity, frequency, and pattern at a specific location according to the ultrasonic radiation signal.

Furthermore, the second processing unit 152 may output a 2-1 ultrasonic radiation signal when the vehicle travels at a constant speed within a preset speed range and output a 2-2 ultrasonic radiation signal when the vehicle performs gear shifting and travels at a speed out of the speed range.

The second processing unit 152 may be configured to determine whether the vehicle stops, travels at a constant speed, performs gear shifting and travels, and a position of the vehicle using vehicle position information (GPS information), vehicle angle information, vehicle speed information, and vehicle acceleration information. The second processing unit 152 may be configured to determine whether the vehicle travels and travels at a constant speed and output ultrasonic radiation signals differently by combining the vehicle position information.

In an exemplary embodiment of the present disclosure, the first output may include a lower ultrasonic signal intensity than the second output. The second processing unit 152 may increase the intensity of the ultrasonic signal to maximize the ultrasonic care effect when the vehicle stops. The second processing unit 152 may be configured for controlling the ultrasonic radiation unit 140 to output a lower ultrasonic signal than in a state in which the vehicle stops to minimize the influence on traveling when the vehicle travels.

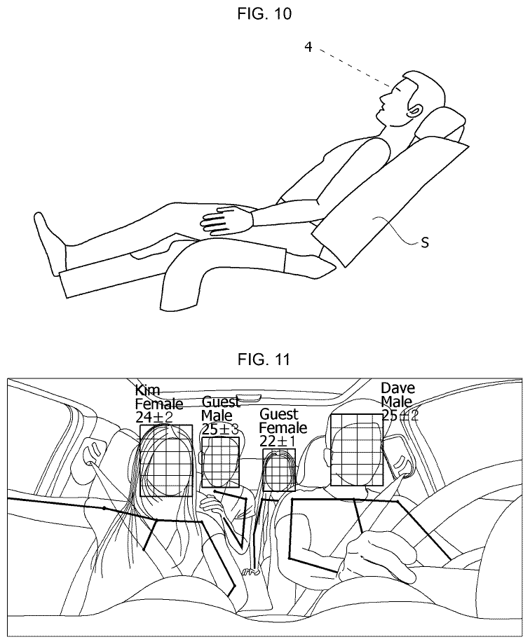

FIG. 4, FIG. 5, FIG. 6, FIG. 7, FIG. 8, FIG. 9, FIG. 10 and FIG. 11 are views for describing an operation of a second processing unit according to an exemplary embodiment of the present disclosure. Referring to FIG. 4 together, the second processing unit 152 may differently set an ultrasonic output position of the first ultrasonic radiation signal and an ultrasonic output position of the second ultrasonic radiation signal. The second processing unit 152 can maximize the ultrasonic care effect by controlling the ultrasonic waves to be directly radiated (solid line) to a front surface of the occupant's face when the vehicle stops. On the other hand, the second processing unit 152 may be configured for controlling the ultrasonic radiation unit 140 to output the ultrasonic wave signal to a vehicle's glass surface G rather than the front surface of the occupant's face to minimize the influence on traveling when the vehicle travels. The ultrasonic waves output to the vehicle's glass are reflected from a glass surface G and indirectly radiated (dotted line) to the occupant's face. Therefore, the second processing unit 152 may adjust a directivity angle of the ultrasonic radiation unit 140 using the position of the vehicle occupant's face and the reflective angle at which the ultrasonic signal is reflected from the glass surface G so that the reflected ultrasonic signal reflected from the vehicle's glass surface G is incident on the front or side surface of the occupant's face.

In an exemplary embodiment of the present disclosure, the first ultrasonic radiation signal may have a fixed frequency value, and the second ultrasonic radiation signal may have a variable frequency value.

In addition, in an exemplary embodiment of the present disclosure, the first ultrasonic signal may have a composite modulation waveform (multi sweep control).

In an exemplary embodiment of the present disclosure, the 2-2 ultrasonic radiation signal may have a lower ultrasonic signal intensity than the 2-1 ultrasonic radiation signal.

In addition, in an exemplary embodiment of the present disclosure, the 2-1 ultrasonic radiation signal may have a double side band (DSB) waveform (sweep control), and the 2-2 ultrasonic radiation signal may have a single side band (SSB) waveform (sweep).

Referring to FIG. 5 together, the second processing unit 152 may output the first ultrasonic radiation signal fixed to a specific frequency ranging from 20 KHz to 30 KHz when the vehicle stops. Human skin accelerates lamellar secretion within an ultrasonic frequency range of about 20 KHz to 30 KHz, which induces skin disease treatment. Therefore, the second processing unit 152 can maximize the customized care effect by outputting ultrasonic waves including a fixed frequency of a specific band when the vehicle stops.

Referring to FIG. 6 together, the second processing unit 152 may be configured for controlling the frequency of the ultrasonic signal to vary between 20 KHz and 30 KHz when the vehicle travels. When the fixed frequency signal is intensively radiated to a person's face, the occupant can feel uncomfortable and driving performance may be affected thereby. Therefore, the second processing unit 152 can minimize the influence on traveling by controlling the frequency of the output ultrasonic waves to vary when the vehicle travels.

Referring to FIG. 7 together, the second processing unit 152 may be configured for controlling the ultrasonic radiation unit 140 to output the ultrasonic signal of the DSB waveform when the vehicle travels at a constant speed within the preset speed range.

Referring to FIG. 8 together, the second processing unit 152 may be configured for controlling the ultrasonic radiation unit 140 to output the ultrasonic signal of the SSB waveform when the vehicle performs gear shifting and travels at a speed out of the preset speed range.

That is, when the vehicle travels at a constant speed on a highway, a country road, etc., the second processing unit 152 may be configured for controlling the ultrasonic radiation unit 140 to output the DSB waveform with a relatively lower output voltage than a composite modulation waveform, and when the vehicle travels in a stationary state in a city, downtown, etc., the second processing unit 152 can minimize the influence on traveling by controlling the ultrasonic radiation unit 140 to output the SSB waveform with a lower output voltage than the DSB waveform.

Referring to FIG. 9 together, the second processing unit 152 may be configured for controlling the ultrasonic radiation unit 140 to output an ultrasonic signal of a composite modulation waveform with a high density and a maximized output voltage through input waves in an SSB and a DSB using an envelope detector when the vehicle stops. Therefore, it is possible to maximize the skin care effect of the occupant through ultrasonic waves. However, when the ultrasonic wave of the complex modulation waveform is intensively radiated to the vehicle occupant, the vehicle occupant may feel uncomfortable. Therefore, as described above, the second processing unit 152 may be configured for controlling the ultrasonic radiation unit 140 to output the ultrasonic signal with the relatively low output voltage when the vehicle travels.

The second processing unit 152 may adjust at least one of an intensity, a frequency, and an ultrasonic output position of the ultrasonic radiation signal using vehicle interior temperature information and vehicle interior humidity information.

Human skin is sensitive to changes in temperature, and when the temperature rises by 1° C., the activity of sebaceous glands increases by 10%, which causes skin trouble by clogging pores due to excessive sebum secretion in summer and causes the skin texture to dry out and lose elasticity due to decreased sebum secretion in winter, resulting in fine wrinkles. For example, an indoor temperature suitable for the skin in winter ranges from 18 to 20° C., and an indoor humidity ranges from 40 to 50%.

Therefore, the second processing unit 152 may adjust the vehicle interior temperature and the vehicle interior humidity by comparing vehicle interior temperature information and vehicle interior humidity information with a preset suitable temperature range and humidity range, increasing the ultrasonic radiation signal when they are out of the suitable temperature range and humidity range, moving the frequency band to a 20 KHz band, or directly radiating the ultrasonic output to the front surface of the face.

Referring to FIG. 10 together, the second processing unit 152 may be configured to determine the occupant's face position using image data, and tilt information and pressure information of a seat S. In an exemplary embodiment of the present disclosure, since the image data may include only upper body information, the second processing unit 152 may be configured to determine the orientation of the face additionally using the tilt information and the pressure information of the seat S. The second processing unit 152 may estimate a direction and degree of tilt of the occupant's body according to the tilt information and the pressure information of the seat S and combine the estimated result with the face position determined through the image data to accurately determine the orientation of the occupant's face. The second processing unit 152 may be configured for controlling an output direction of the ultrasonic radiation unit 140 based on the face position and orientation of the occupant.

Referring to FIG. 11, the second processing unit 152 may adjust at least one of an intensity, a frequency, and an ultrasonic output position of the ultrasonic radiation signal according to the number of vehicle occupants.

For example, the second processing unit 152 may strongly adjust the intensity of the ultrasonic radiation signal when more occupants are in a vehicle as compared to a case where only a driver is in the vehicle.

For example, the second processing unit 152 may be configured for controlling the ultrasonic radiation unit 140 to output the ultrasonic signal at a variable frequency when a plurality of occupants are in the vehicle.

For example, the second processing unit 152 may be configured for controlling the ultrasonic radiation unit 140 to output ultrasonic waves while rotating when the plurality of occupants are in the vehicle.

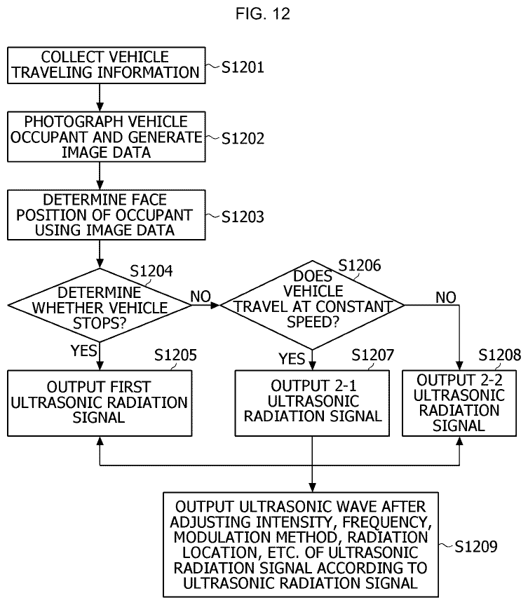

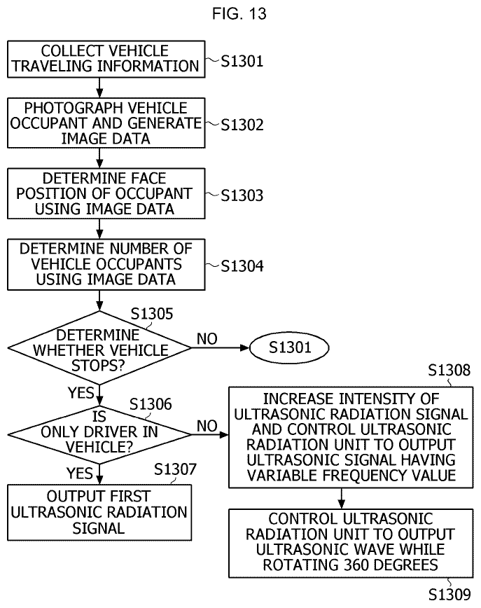

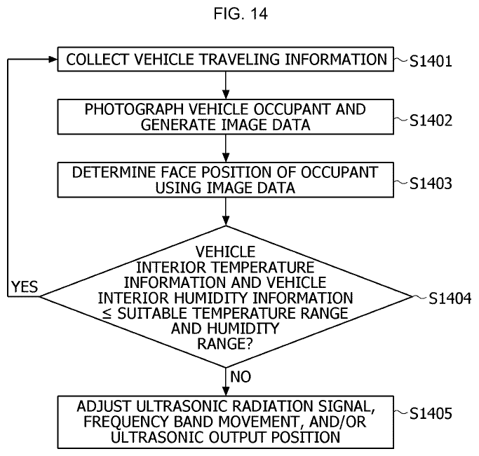

FIG. 12 is a flowchart of a vehicle occupant care method according to an exemplary embodiment of the present disclosure.

Referring to FIG. 12, first, the communication unit collects traveling information of a vehicle (S1201).

Next, the camera photographs a vehicle occupant and generates image data (S1202).

Next, the first processing unit is configured to determine the face position of the occupant using the image data (S1203).

Next, the second processing unit is configured to determine whether the vehicle stops based on the traveling information (S1204).

Next, the second processing unit outputs the first ultrasonic radiation signal when the vehicle stops (S1205). The first ultrasonic radiation signal may have the fixed frequency value of 20 KHz and have the composite modulation waveform (multi sweep control). The first ultrasonic radiation signal sets the front surface of the occupant's face to the ultrasonic output position.

When the vehicle does not stop, the second processing unit is configured to determine whether the vehicle travels at a constant speed (S1206).

When the vehicle travels at a constant speed, the second processing unit outputs the 2-1 ultrasonic radiation signal (S1207). The 2-1 ultrasonic radiation signal may have a variable frequency value ranging from 20 KHz to 30 KHz and have a DSB waveform. The 2-1 ultrasonic radiation signal sets the ultrasonic output position so that the ultrasonic wave reflected from the vehicle glass surface may be incident on the front or side surface of the occupant's face.

Alternatively, when the vehicle does not travel at a constant speed, the second processing unit outputs the 2-2 ultrasonic radiation signal (S1208). The 2-2 ultrasonic radiation signal may have a variable frequency value ranging from 20 KHz to 30 KHz and have a SSB waveform. The 2-2 ultrasonic radiation signal sets the ultrasonic output position so that the ultrasonic wave reflected from the vehicle glass surface may be incident on the front or side surface of the occupant's face.

The ultrasonic radiation unit outputs ultrasonic waves by adjusting the intensity, frequency, modulation method, radiation position, etc. of the ultrasonic radiation signal according to the ultrasonic radiation signal received from the second processing unit (S1209).

FIG. 13 is a flowchart of a vehicle occupant care method according to another exemplary embodiment of the present disclosure.

Referring to FIG. 13, first, the communication unit collects traveling information of a vehicle (S1301).

Next, the camera photographs a vehicle occupant and generates image data (S1302).

Next, the first processing unit is configured to determine the face position of the occupant using the image data (S1303).

Next, the first processing unit is configured to determine the number of vehicle occupants using the image data (S1304).

The second processing unit is configured to determine whether the vehicle is in a stopped state using the traveling information (S1305).

When the vehicle is in the stopped state and only a driver is in the vehicle, the second processing unit outputs the first ultrasonic radiation signal (S1306, S1307). The first ultrasonic radiation signal may have the fixed frequency value of 20 KHz and have the composite modulation waveform (multi sweep control). The first ultrasonic radiation signal sets the front surface of the occupant's face to the ultrasonic output position.

When the vehicle is in the stopped state and a plurality of occupants are in the vehicle, the second processing unit increases the intensity of the ultrasonic radiation signal and is configured to control the ultrasonic radiation unit to output the ultrasonic signal including the variable frequency value ranging from 20 KHz to 30 KHz (S1308).

In the instant case, the second processing unit is configured to control the ultrasonic radiation unit to output ultrasonic waves while rotating 360 degrees (S1309).

FIG. 14 is a flowchart of a vehicle occupant care method according to yet another exemplary embodiment of the present disclosure.

Referring to FIG. 14, first, the communication unit collects traveling information of a vehicle (S1401).

Next, the camera photographs a vehicle occupant and generates image data (S1402).

Next, the first processing unit is configured to determine the face position of the occupant using the image data (S1403).

Next, the second processing unit compares vehicle interior temperature information and vehicle interior humidity information with the preset suitable temperature range (S1404).

Next, when the vehicle interior temperature information and the vehicle interior humidity information are out of the suitable temperature range and humidity range, the second processing unit adjusts vehicle interior temperature and vehicle interior humidity by increasing the ultrasonic radiation signal, moving the frequency band to the 20 kHz band, or directly radiating the ultrasonic output to the front surface of the face. In the instant case, the second processing unit adjusts the intensity and frequency band of the ultrasonic radiation signal, the time for which ultrasonic waves are radiated to the front surface of the face, etc. in proportion to the degree to which the vehicle interior temperature information and the vehicle interior humidity information are out of the suitable temperature range and humidity range (S1405).

A vehicle occupant care device and method according to an exemplary embodiment can provide an optimized skin care solution for a vehicle occupant in a vehicle interior.

Furthermore, it is possible to provide a differentiated skin care solution depending on a traveling situation of a vehicle.

Furthermore, it is possible to provide an optimized skin care solution depending on the number of vehicle occupants.

Furthermore, it is possible to provide a skin care solution through voice instruction without direct manipulation of a device.

Therefore, safe driving may be maintained.

In various exemplary embodiments of the present disclosure, the memory and the processor may be provided as one chip, or provided as separate chips.

In various exemplary embodiments of the present disclosure, the scope of the present disclosure includes software or machine-executable commands (e.g., an operating system, an application, firmware, a program, etc.) for enabling operations according to the methods of various embodiments to be executed on an apparatus or a computer, a non-transitory computer-readable medium including such software or commands stored thereon and executable on the apparatus or the computer.

In various exemplary embodiments of the present disclosure, the control device may be implemented in a form of hardware or software, or may be implemented in a combination of hardware and software.

Software implementations may include software components (or elements), object-oriented software components, class components, task components, processes, functions, attributes, procedures, subroutines, program code segments, drivers, firmware, microcode, data, database, data structures, tables, arrays, and variables. The software, data, and the like may be stored in memory and executed by a processor. The memory or processor may employ a variety of means well-known to a person including ordinary knowledge in the art.

Furthermore, the terms such as “unit”, “module”, etc. included in the specification mean units for processing at least one function or operation, which may be implemented by hardware, software, or a combination thereof.

In the flowchart described with reference to the drawings, the flowchart may be performed by the controller or the processor. The order of operations in the flowchart may be changed, a plurality of operations may be merged, or any operation may be divided, and a specific operation may not be performed. Furthermore, the operations in the flowchart may be performed sequentially, but not necessarily performed sequentially. For example, the order of the operations may be changed, and at least two operations may be performed in parallel.

Hereinafter, the fact that pieces of hardware are coupled operatively may include the fact that a direct and/or indirect connection between the pieces of hardware is established by wired and/or wirelessly.

In an exemplary embodiment of the present disclosure, the vehicle may be referred to as being based on a concept including various means of transportation. In some cases, the vehicle may be interpreted as being based on a concept including not only various means of land transportation, such as cars, motorcycles, trucks, and buses, that drive on roads but also various means of transportation such as airplanes, drones, ships, etc.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The term “and/or” may include a combination of a plurality of related listed items or any of a plurality of related listed items. For example, “A and/or B” includes all three cases such as “A”, “B”, and “A and B”.

In exemplary embodiments of the present disclosure, “at least one of A and B” may refer to “at least one of A or B” or “at least one of combinations of at least one of A and B”. Furthermore, “one or more of A and B” may refer to “one or more of A or B” or “one or more of combinations of one or more of A and B”.

In the present specification, unless stated otherwise, a singular expression includes a plural expression unless the context clearly indicates otherwise.

In the exemplary embodiment of the present disclosure, it should be understood that a term such as “include” or “have” is directed to designate that the features, numbers, steps, operations, elements, parts, or combinations thereof described in the specification are present, and does not preclude the possibility of addition or presence of one or more other features, numbers, steps, operations, elements, parts, or combinations thereof.

According to an exemplary embodiment of the present disclosure, components may be combined with each other to be implemented as one, or some components may be omitted.

The foregoing descriptions of specific exemplary embodiments of the present disclosure have been presented for purposes of illustration and description. They are not intended to be exhaustive or to limit the present disclosure to the precise forms disclosed, and obviously many modifications and variations are possible in light of the above teachings. The exemplary embodiments were chosen and described in order to explain certain principles of the invention and their practical application, to enable others skilled in the art to make and utilize various exemplary embodiments of the present disclosure, as well as various alternatives and modifications thereof. It is intended that the scope of the present disclosure be defined by the Claims appended hereto and their equivalents.

What is claimed is:

1. A vehicle occupant care apparatus comprising:
a communication unit configured to collect traveling information of a vehicle;
a camera configured to photograph a vehicle occupant and generate image data related to the vehicle occupant;
a first processing unit configured to determine a face position of the vehicle occupant using the image data;
a second processing unit configured to output a first ultrasonic radiation signal in response that the vehicle stops and output a second ultrasonic radiation signal in response that the vehicle travels according to the traveling information; and
an ultrasonic radiation unit fixed to an interior of the vehicle and configured to output an ultrasonic wave to the face position of the vehicle occupant according to the first ultrasonic radiation signal or the second ultrasonic radiation signal.

2. The vehicle occupant care apparatus of claim 1, wherein the first output includes a lower ultrasonic signal intensity than the second output.

3. The vehicle occupant care apparatus of claim 1, wherein the second processing unit differently sets an ultrasonic output position of the first ultrasonic radiation signal and an ultrasonic output position of the second ultrasonic radiation signal.

4. The vehicle occupant care apparatus of claim 1, wherein the first ultrasonic radiation signal has a fixed frequency value.

5. The vehicle occupant care apparatus of claim 1, wherein the second ultrasonic radiation signal has a variable frequency value.

6. The vehicle occupant care apparatus of claim 1, wherein the first ultrasonic radiation signal has a composite modulation waveform.

7. The vehicle occupant care apparatus of claim 1, wherein the second processing unit outputs a 2-1 ultrasonic radiation signal in response that the vehicle travels at a constant speed within a preset speed range.

8. The vehicle occupant care apparatus of claim 7, wherein the second processing unit outputs a 2-2 ultrasonic radiation signal in response that the vehicle performs gear shifting and travels at a speed out of the preset speed range.

9. The vehicle occupant care apparatus of claim 8, wherein the 2-2 ultrasonic radiation signal has a lower ultrasonic signal intensity than the 2-1 ultrasonic radiation signal.

10. The vehicle occupant care apparatus of claim 7, wherein the 2-1 ultrasonic radiation signal has a double side band (DSB) waveform.

11. The vehicle occupant care apparatus of claim 8, wherein the 2-2 ultrasonic radiation signal has a single side band (SSB) waveform.

12. The vehicle occupant care apparatus of claim 1,
wherein the occupant is in plural,
wherein the first processing unit is configured to determine a number of the vehicle occupants using the image data, and
wherein the second processing unit adjusts at least one of an intensity, a frequency, and an ultrasonic output position of an ultrasonic radiation signal according to the number of the vehicle occupants.

13. The vehicle occupant care apparatus of claim 1, wherein the communication unit collects tilt information and pressure information of a seat on which the vehicle occupant sits.

14. The vehicle occupant care apparatus of claim 13, wherein the second processing unit is configured to determine the face position of the vehicle occupant using the image data, and the tilt information and the pressure information of the seat.

15. A vehicle occupant care method including:
collecting, by a communication unit, traveling information of a vehicle;
photographing, by a camera, a vehicle occupant and generating image data related to the vehicle occupant;
determining, by a first processing unit, a face position of the vehicle occupant using the image data;
determining, by a second processing unit, whether the vehicle stops based on the traveling information;
outputting, by the second processing unit, a first ultrasonic radiation signal in response that the vehicle stops or outputting a second ultrasonic radiation signal in response that the vehicle travels; and
outputting, by an ultrasonic radiation unit fixed to an interior of the vehicle, an ultrasonic wave to the face position of the vehicle occupant according to the first ultrasonic radiation signal or the second ultrasonic radiation signal.

16. The vehicle occupant care method of claim 15,
wherein the first output includes a lower ultrasonic signal intensity than the second output.

17. The vehicle occupant care method of claim 15, wherein the outputting of the second ultrasonic radiation signal includes:
determining, by the second processing unit, whether the vehicle travels at a constant speed; and
outputting, by the second processing unit, a 2-1 ultrasonic radiation signal in response that the vehicle travels at the constant speed.

18. The vehicle occupant care method of claim 17, further including outputting, by the second processing unit, a 2-2 ultrasonic radiation signal in response that the vehicle performs gear shifting and travels.

19. The vehicle occupant care method of claim 18, wherein the 2-2 ultrasonic radiation signal has a lower ultrasonic signal intensity than the 2-1 ultrasonic radiation signal.

20. The vehicle occupant care method of claim 15,
wherein the occupant is in plural, and
wherein the vehicle occupant care method further includes:
determining, by the first processing unit, a number of the vehicle occupants using the image data; and
adjusting, by the second processing unit, at least one of an intensity, a frequency, and an ultrasonic output position of an ultrasonic radiation signal according to the number of the vehicle occupants.
